(19) United States
(12) Patent Application Publication
(10) Pub. No.: US 2025/0352744 A1
(43) Pub. Date: Nov. 20, 2025

(54) WEARABLE INHALATION DEVICE WITH MODULAR POD SYSTEM

(71) Applicant: Wrip Inc., San Diego, CA (US)

(72) Inventor: Zion Shepherd, San Deigo, CA (US)

(21) Appl. No.: 19/048,063

(22) Filed: Feb. 7, 2025

Related U.S. Application Data
(60) Provisional application No. 63/649,910, filed on May 20, 2024.

(51) Int. Cl. A61M 15/00

(52) U.S. Cl. CPC A61M 15/0028; A61M 15/0021; A61M 2202/0007; A61M 2205/12; A61M 2205/18; A61M 2205/3303; A61M 2205/3327; A61M 2205/3334; A61M 2205/3368; A61M 2205/3592; A61M 2205/3653; A61M 2205/502; A61M 2205/8206; A61M 2209/088; A61M 2210/083; A61M 2230/06; A61M 2230/20; A61M 2230/50

(57) ABSTRACT

The present disclosure provides a wearable inhalation device including a housing configured to be worn on a user's wrist. The housing includes a chamber for receiving a substance pod. The device includes a modular pod system within the housing configured to interchangeably receive substance pods containing various substances. A sensor is configured to detect the suction of the user's breath and activate the device. A heating element is configured to vaporize the substance within the substance pod upon activation. A mouthpiece in fluid communication with the chamber is configured to deliver the vaporized substance to the user upon inhalation.

### CROSS-REFERENCE TO RELATED APPLICATIONS

This application claims priority from U.S. Provisional Application Ser. No. 63/649,910 filed on May 20, 2024, which is incorporated herein by reference in its entirety.

### STATEMENT REGARDING FEDERALLY SPONSORED RESEARCH OR DEVELOPMENT

Not Applicable.

### FIELD

The present disclosure relates to wearable devices for substance administration, and more particularly to a wrist-worn inhalation device with a modular pod system for discreet and portable delivery of vaporized substances.

### INTRODUCTION

Inhalation devices are commonly used for administering various substances, including medicinal and recreational substances. These devices typically work by heating a substance to create a vapor, which is then inhaled by the user. The substance can be in various forms, such as liquids, concentrates, waxes, or dry materials. The heating element within the device is typically powered by a battery and can be activated manually or automatically.

One type of inhalation device is a vaporizer, which is often used for consuming substances like nicotine, cannabidiol (CBD), tetrahydrocannabinol (THC), and other herbal extracts. Vaporizers are considered an alternative to traditional smoking methods as they heat the substance to a temperature that releases the active compounds without combustion, thereby reducing the production of harmful byproducts.

Inhalation devices are typically standalone units that are carried separately by the user. They come in various sizes and designs, ranging from large, stationary units to small, portable devices. Portable inhalation devices, such as vape pens and e-cigarettes, are designed for convenience and mobility, allowing users to carry and use them almost anywhere.

However, due to their small size, portable inhalation devices can be easily misplaced or lost. Furthermore, using these devices in public can sometimes attract unwanted attention or judgment due to the visible vapor they produce and the stigma associated with vaping or smoking.

Wearable technology has become increasingly popular in recent years, with devices like smartwatches and fitness trackers becoming commonplace. These devices are typically worn on the wrist and offer various functionalities, such as tracking physical activity, monitoring health metrics, and providing notifications from a connected smartphone.

Inhalation devices and wearable technology are generally considered separate categories of products, each serving distinct purposes. However, there is a growing interest in combining different functionalities into a single device to enhance convenience and usability. This has led to the development of multifunctional devices that integrate various features into a compact and portable form factor.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a wearable inhalation device is provided. The device includes a housing configured to be worn on a user's wrist, the housing including a chamber for receiving a substance pod. The device also includes a modular pod system within the housing, which is configured to interchangeably receive substance pods containing various substances. The device further includes a sensor configured to detect the suction of the user's breath and activate the device, a heating element configured to vaporize the substance within the substance pod upon activation, and a mouthpiece in fluid communication with the chamber. The mouthpiece is configured to deliver the vaporized substance to the user upon inhalation.

According to other aspects of the present disclosure, the device may include one or more of the following features. The housing may be integrated into a wristband, configured to secure the device to the user's wrist. The sensor may be an airflow sensor configured to detect the suction of the user's breath by sensing a change in air pressure within the device. The heating element may be a coil configured to heat the substance within the substance pod to a predetermined temperature to vaporize the substance. The substance pod may be configured to contain a liquid substance, selected from the group consisting of CBD, THC, melatonin, caffeine, and combinations thereof. The device may further include a power source within the housing, configured to supply electrical power to the heating element. The power source may be a rechargeable battery. The device may also include a control mechanism, configured to allow the user to adjust the intensity of the vapor produced by the device. The modular pod system may be configured to receive substance pods of different sizes, each size corresponding to a different quantity of substance.

According to another aspect of the present disclosure, a method for administering substances using a wearable inhalation device is provided. The method includes providing a wearable inhalation device having a housing configured to be worn on a user's wrist, the housing including a chamber for receiving a substance pod. The method further includes inserting a selected substance pod into the modular pod system of the device, the substance pod containing a desired substance. The method also includes activating the device in response to detecting the suction of the user's breath via a sensor, vaporizing the substance within the substance pod using a heating element, and delivering the vaporized substance to the user through a mouthpiece in fluid communication with the chamber upon inhalation.

According to other aspects of the present disclosure, the method may include one or more of the following features. The substance pod may contain a substance selected from the group consisting of CBD, THC, melatonin, caffeine, and combinations thereof. The method may include a step of recharging a power source within the housing, the power source configured to supply electrical power to the heating element. The sensor may be an airflow sensor configured to detect the suction of the user's breath by sensing a change in air pressure within the device. The method may further include a step of adjusting the intensity of the vapor produced by the device using a control mechanism. The step of inserting a selected substance pod into the modular pod system of the device may include selecting a substance pod of a specific size, each size corresponding to a different quantity of substance.

According to yet another aspect of the present disclosure, a wearable inhalation device is provided. The device includes a housing configured to be worn on a user's wrist, the housing including a chamber for receiving a substance pod. The device also includes a modular pod system within the housing, the modular pod system configured to interchangeably receive substance pods containing various substances. The device further includes a sensor configured to detect the suction of the user's breath and activate the device, a heating element configured to vaporize the substance within the substance pod upon activation, a mouthpiece in fluid communication with the chamber, and a smart feature. The mouthpiece is configured to deliver the vaporized substance to the user upon inhalation, and the smart feature is configured to provide health tracking functionalities and substance usage data to the user.

According to other aspects of the present disclosure, the smart feature may be configured to track and display the user's substance usage data, including the type of substance used, the quantity of substance used, and the frequency of use. The smart feature may also be configured to provide health tracking functionalities, including tracking the user's heart rate, oxygen level, and body temperature. The smart feature may further be configured to provide alerts to the user based on the health tracking data, the alerts including reminders to administer the substance at predetermined intervals or warnings when abnormal health data is detected.

The foregoing general description of the illustrative embodiments and the following detailed description thereof are merely exemplary aspects of the teachings of this disclosure and are not restrictive.

### DRAWINGS

Those of skill in the art will understand that the drawings, described below, are for illustrative purposes only. The drawings are not intended to limit the scope of the present teachings in any way.

FIGS. 1 and 2 illustrate isometric views of a wearable inhalation device from different angles, according to aspects of the present disclosure.

FIG. 3 illustrates an exploded view of a wearable inhalation device, according to an embodiment.

FIG. 4 illustrates an exploded view of a wearable inhalation device, according to aspects of the present disclosure.

FIG. 5 illustrates an exploded view of a wearable inhalation device, according to an embodiment.

FIGS. 6-7 illustrate exploded and perspective views of a circuit board assembly for a wearable inhalation device, according to aspects of the present disclosure.

FIG. 7 illustrates a section view of the wearable inhalation device, according to an embodiment.

FIG. 8 illustrates an isometric view of a wearable inhalation device, according to aspects of the present disclosure.

FIGS. 9-10 illustrate different views of a wearable device, according to an embodiment.

FIG. 11 illustrates an exploded view of a substance pod assembly for a wearable inhalation device, according to aspects of the present disclosure.

FIG. 12 illustrates an orthogonal side view showing an exemplary wrist position of a wearable device, according to an embodiment.

FIG. 13 illustrates a side view of a person using a wrist-mounted inhalation device, according to aspects of the present disclosure.

FIGS. 14-15 illustrate isometric views of a wearable inhalation device from different angles, according to an embodiment.

FIGS. 16 and 17 illustrate isometric views of a wearable device from different angles, according to aspects of the present disclosure.

FIG. 18 illustrates an exploded view of a wearable inhalation device, according to an embodiment.

FIG. 19 illustrates an exploded view of a wearable inhalation device, according to aspects of the present disclosure.

FIG. 20 illustrates an exploded view of a wearable inhalation device, according to an embodiment.

FIG. 21 illustrates a top orthogonal view of a wearable inhalation device, according to aspects of the present disclosure.

FIG. 22 illustrates a section view of a wearable inhalation device, according to an embodiment.

FIG. 23 illustrates an isometric view of a wearable device, according to aspects of the present disclosure.

### DETAILED DESCRIPTION

### Abbreviations and Definitions

To facilitate understanding of the invention, a number of terms and abbreviations as used herein are defined in the following Table as follows:

TABLE 1
Labeled Device Parts

housing cover: 101
pod housing: 102
pod assembly: 102a
airflow port: 103
charging port: 104
band connector: 105
control button: 106
indicator opening: 107
main enclosure: 108
power cell: 109
circuit board: 110
charging port: 110a
sensor module: 110b
circuit components: 110c
indicator light: 110d
circuit board cover: 110e
airflow seal: 111
first magnet: 112a
second magnet: 112b
first male connector pin: 113a
second male connector pin: 113b
housing seal: 114
first female connector pin: 115a
second female connector pin: 115b
pod base: 116
alignment magnet: 117
heating coil: 118
ceramic element: 119
coil cover: 120
heating chamber: 121
substance reservoir: 122
reservoir base: 123
reservoir housing: 124
pod seal: 125
sensor ring: 126
button housing: 127
wearable band: 129
exemplary wrist position: 135
contact assembly: 150
circuit housing: 151
base plate: 152
support feet: 153
mounting bracket: 155

Wearable Inhalation Device with Modular Pod System

The following description sets forth exemplary aspects of the present disclosure. It should be recognized, however, that such description is not intended as a limitation on the scope of the present disclosure. Rather, the description also encompasses combinations and modifications to those exemplary aspects described herein.

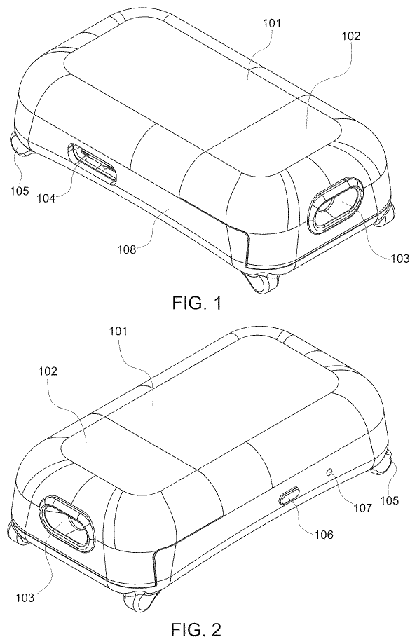

The wearable inhalation device may be designed for substance administration through inhalation while being worn on a user's wrist. FIGS. 1 and 2 illustrate isometric views of the wearable inhalation device from different angles, showing its external appearance and main features.

As shown in FIGS. 1 and 2, the wearable inhalation device may include a housing cover 101 that forms the outer shell of the device. In some cases, the housing cover 101 may be made of skin-safe materials with varying textures to ensure user comfort and safety during prolonged wear.

The device may also include a pod housing 102 that contains components for substance delivery. In some implementations, the pod housing 102 may be transparent, solid, or a combination of both, allowing for visual inspection of the internal components or substance levels as needed.

An airflow port 103 may be positioned on one end of the device to facilitate air movement during operation. The device may also include a charging port 104 located on one side, which enables power replenishment.

A band connector 105 may be integrated into the design to allow attachment to a wearable band. In some cases, the wearable band may be made of different materials including silicone, metal, or other commercially viable alternatives, providing options for user comfort and style preferences.

A control button 106 may be positioned on the side of the device for user operation, and an indicator opening 107 may provide visual feedback of device status.

The main enclosure 108 may form the structural foundation of the device and house internal components. In some implementations, the main enclosure 108 may have different shapes including square, circle, rectangle, or other variable shapes, allowing for design flexibility and adaptation to various user preferences.

The overall design of the wearable inhalation device may present a compact form factor with rounded edges and integrated features positioned for accessibility when worn. This configuration may allow for discreet substance administration while maintaining the appearance of a typical wrist-worn accessory.

### Internal Components and Assembly

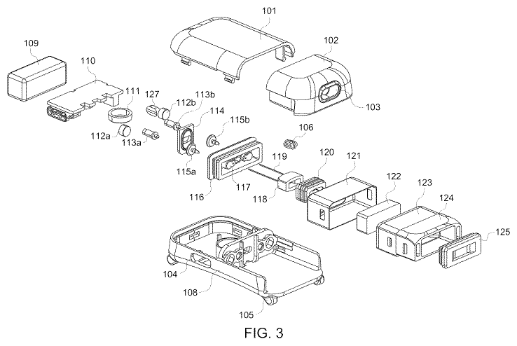

The wearable inhalation device may include various internal components arranged to facilitate substance vaporization and delivery. Referring to FIG. 3, the device may include a power cell 109 positioned within the main enclosure 108. The power cell 109 may provide electrical energy to operate the device's components.

Adjacent to the power cell 109, a circuit board 110 may be located. The circuit board 110 may contain electronic components for controlling the device's functions. An airflow seal 111 may be positioned near the circuit board 110 to manage airflow within the device.

The device may incorporate a magnetic connection system comprising a first magnet 112a and a second magnet 112b. These magnets may work in conjunction with a first male connector pin 113a and a second male connector pin 113b. The male connector pins may interface with a first female connector pin 115a and a second female connector pin 115b, facilitating electrical connections within the device.

A housing seal 114 may be included to provide a barrier between internal components and external elements. The device may also feature a pod base 116 with an alignment magnet 117, which may assist in proper positioning of the substance delivery components.

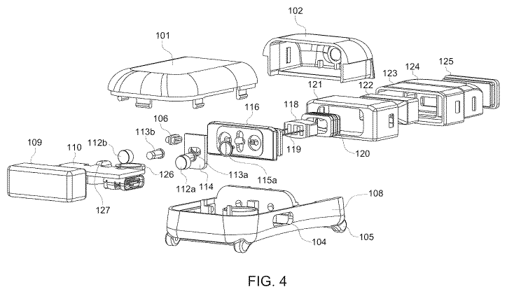

Referring now to FIG. 4, the substance delivery system may include a heating coil 118. In some cases, the heating coil 118 may be made of different materials including stainless steel, nickel, titanium, clapton, mesh, ceramic, cotton, or quartz. Temperature Control Coils can include dual coils, triple coils, quad coils, and standard coils (Kanthal). The heating coil 118 may work in conjunction with a ceramic element 119 to vaporize the substance. A coil cover 120 may enclose these heating components within a heating chamber 121.

The substance to be vaporized may be contained within a substance reservoir 122. The substance reservoir 122 may be positioned between a reservoir base 123 and a reservoir housing 124. A pod seal 125 may ensure proper containment of the substance within the reservoir assembly.

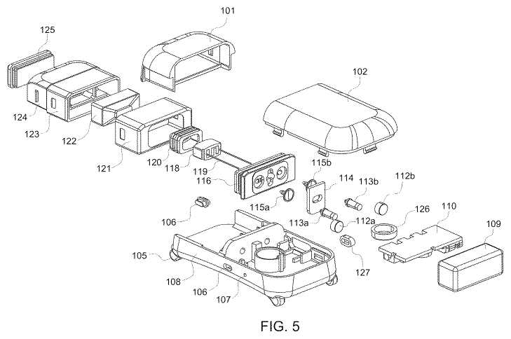

As shown in FIG. 5, the device may also include a button housing 127 to accommodate user controls. A sensor ring 126 may be incorporated to detect user interactions or environmental conditions.

The arrangement of these components within the main enclosure 108 may allow for efficient assembly and operation of the wearable inhalation device. The modular design may facilitate maintenance and replacement of individual components as needed.

### Circuit Board Assembly

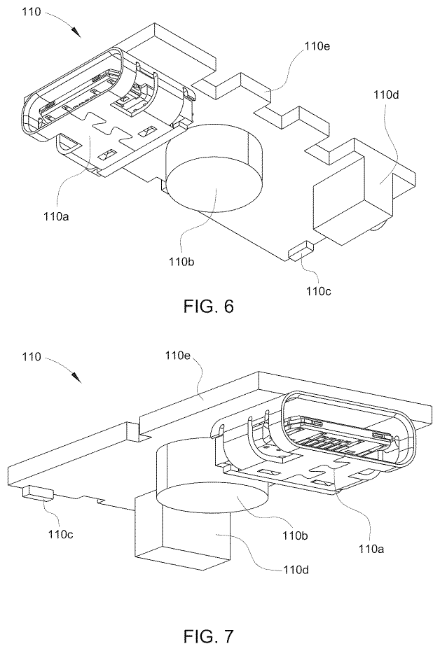

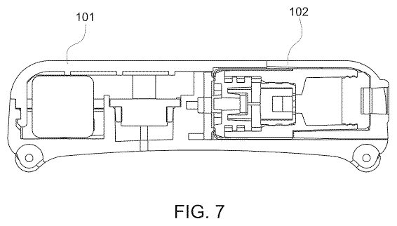

FIGS. 6-7 illustrate exploded and perspective views of a circuit board assembly for the wearable inhalation device. The circuit board assembly may include a circuit board 110 that serves as the main electronic component platform. The circuit board 110 may include several integrated elements arranged in a compact configuration.

A charging port 110a may be positioned at one end of the circuit board 110, providing a connection point for power input. In some cases, the charging port 110a may be configured to use different charging methods. These methods may include USB, wireless, magnetic, kinetic, or heat transfer charging, allowing for flexibility in how the device is recharged.

Adjacent to the charging port 110a, a sensor module 110b may be integrated into the circuit board assembly. The sensor module 110b may be depicted as a cylindrical component in FIGS. 6-7. This sensor module 110b may be responsible for detecting user interactions or environmental conditions relevant to the device's operation.

Circuit components 110c may be arranged on the circuit board 110 and provide the electronic functionality for the device. These circuit components 110c may include various electronic elements such as microprocessors, memory chips, and other necessary components for controlling the device's functions.

An indicator light 110d may be incorporated into the assembly. The indicator light 110d may be positioned to be visible when the device is assembled, potentially aligning with the indicator opening 107 in the housing cover 101. This indicator light 110d may provide visual feedback to the user about the device's status, such as power levels or operational modes.

A circuit board cover 110e may extend over the circuit board 110, providing protection for the electronic components while allowing access to necessary connection points. This circuit board cover 110e may help maintain the integrity of the electronic components and shield them from external factors.

The arrangement of these components on the circuit board 110 may be designed to create a compact and integrated assembly. This configuration may allow for efficient use of space within the main enclosure 108 of the wearable inhalation device, contributing to its overall compact form factor.

### Device Housing and Wearable Configuration

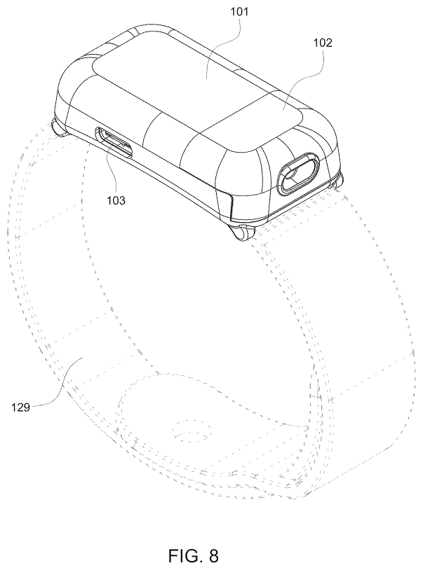

The wearable inhalation device may be designed to be worn on a user's wrist, providing a discreet and convenient method for substance administration. Referring to FIG. 8, the device may include a housing cover 101 that forms the main exterior structure. The pod housing 102 may be integrated into the housing cover 101 and may contain components for substance delivery.

In some cases, an airflow port 103 may be positioned on the device to enable air passage during operation. The airflow port 103 may be placed at different angles, including on the side of the pod housing 102 or near the edge of the pod housing 102, allowing for flexibility in design and user preference.

The device may be attached to a wearable band 129, which may enable the device to be worn on a user's wrist. The wearable band 129 may connect to the housing cover 101 to form a complete wearable unit. In some implementations, the components may be arranged in a compact configuration that allows for portability while maintaining functionality.

FIG. 7 illustrates a section view of the wearable inhalation device, showing the internal configuration and arrangement of components. The section view may reveal the internal structure of the housing cover 101 and pod housing 102. The housing cover 101 may form the outer shell of the device and may contain various internal compartments and channels. The pod housing 102 may be positioned at one end of the device and may include internal features for accommodating the substance delivery components.

Referring to FIG. 8, the wearable band 129 may be shown in dashed lines, indicating its connection to the housing cover 101. This configuration may allow the device to be securely worn on the user's wrist while maintaining access to the airflow port 103 for substance administration.

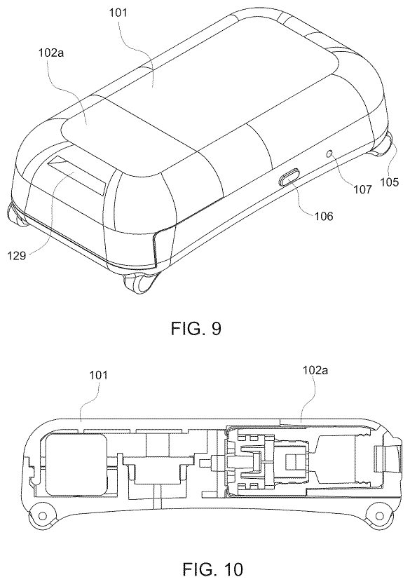

In some cases, the device may incorporate a pod assembly 102a, as shown in FIG. 9. The pod assembly 102a may be integrated into the housing cover 101 and may contain the components necessary for substance vaporization and delivery. The device may include a band connector 105 for attaching the wearable band 129, ensuring a secure fit on the user's wrist.

The control button 106 may be positioned on the side of the housing cover 101, allowing for easy access and operation while the device is worn. An indicator opening 107 may be provided for displaying device status information, enhancing user interaction with the device.

FIGS. 9-10 depict two views of a wearable inhalation device, providing different perspectives on its design and features. In FIG. 9, an isometric view of the device is presented, highlighting the watch body cover 101 and the pod cover and mouthpiece 102a. The watch body cover 101 serves as the main structural component of the device, providing a protective shell for the internal elements and contributing to the overall aesthetic. The pod cover and mouthpiece 102a is positioned on the upper part of the watch body cover 101 and is designed to house the mechanisms for vaporizing and delivering the substance to the user.

The watchband attachment mechanism 105 is located on the side of the device, enabling the device to be secured to the user's wrist. This feature allows for convenience and ensures that the device remains easily accessible for the user. The power and action button 106 is also situated on the side of the device, providing a means for the user to manually activate or control various functions of the device.

The LED indicator 107 is strategically placed on the front face of the device, providing visual feedback to the user regarding the device's status, such as battery life, readiness for use, or connectivity with other devices. This visual feedback is an integral part of the user interface, enhancing the usability and interactive experience of the device.

In FIG. 10, a bottom view of the device is presented, revealing the wristband 129, which is attached to the watch body cover 101. The wristband 129 is designed to comfortably and securely hold the device on the user's wrist, facilitating the wearable nature of the inhalation device. The materials and design of the wristband 129 may be chosen to provide durability and comfort against the skin, making the device suitable for extended wear.

The pod cover and mouthpiece 102a, visible in both FIGS. 9 and 10, is an integral part of the device that contains the control elements for operation. This component is likely to include elements such as the airflow sensor, heating mechanism, and substance reservoir, which work in conjunction to facilitate the inhalation process when the device is activated by the user.

Together, FIGS. 9-10 provide a comprehensive view of the external features of the wearable inhalation device, illustrating the careful design and arrangement of the components that contribute to the device's functionality and user experience. The views depicted in these figures highlight the compact and integrated design of the device, which is both functional and discreet, aligning with the objectives of providing a wearable device for substance administration that can seamlessly integrate into the user's daily routine.

The wearable configuration of the device may allow for discreet substance administration while maintaining the appearance of a typical wrist-worn accessory. This design may enable users to integrate the device seamlessly into their daily routines, providing convenience and privacy during use.

### Substance Pod Assembly

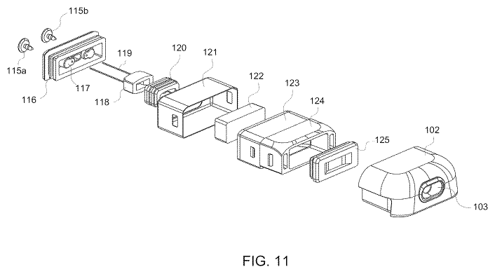

The wearable inhalation device may include a modular substance pod assembly that facilitates the vaporization and delivery of substances. FIG. 11 illustrates an exploded view of the substance pod assembly, showing the various components and their spatial relationships.

The pod housing 102 may form the outer shell of the assembly and may include the airflow port 103. In some cases, the airflow port 103 may be positioned to allow for optimal air intake or exhaust during the vaporization process.

The first female connector pin 115a and the second female connector pin 115b may be positioned at one end of the assembly. These connector pins may interface with the corresponding male connector pins in the main device, establishing electrical connections for power and control signals.

The pod base 116 may be connected to the female connector pins and may incorporate the alignment magnet 117. In some implementations, the alignment magnet 117 may work in conjunction with magnets in the main device to ensure proper positioning and secure attachment of the substance pod assembly.

The heating coil 118 may be positioned adjacent to the pod base 116. In some cases, the heating coil 118 may be made of various materials such as stainless steel, nickel, titanium, or ceramic, depending on the specific vaporization requirements of the substance.

The ceramic element 119 may be located next to the heating coil 118. The ceramic element 119 may provide additional heat distribution or act as a surface for substance vaporization. The coil cover 120 may enclose the heating coil 118 and ceramic element 119, potentially providing protection and directing heat flow.

The heating chamber 121 may house the heating components, creating a controlled environment for substance vaporization. The heating chamber 121 may be designed to efficiently transfer heat to the substance while minimizing heat loss to other parts of the device.

The substance reservoir 122 may be positioned above the heating components. In some implementations, the substance reservoir 122 may be designed to hold various types of substances, such as liquids, waxes, or dry materials, depending on the intended use of the device.

The reservoir base 123 and reservoir housing 124 may form the containment structure for the substance to be vaporized. These components may be designed to securely hold the substance while allowing for efficient heat transfer from the heating elements.

The pod seal 125 may provide a sealing interface between the components, ensuring proper containment of the substance and vapor pathway. In some cases, the pod seal 125 may be made of heat-resistant materials to maintain its integrity during the vaporization process.

The arrangement of these components in the substance pod assembly may allow for efficient vaporization and delivery of the substance. The modular nature of the assembly may facilitate easy replacement or refilling of substances, enhancing the versatility and user-friendliness of the wearable inhalation device.

### Device Placement and Usage

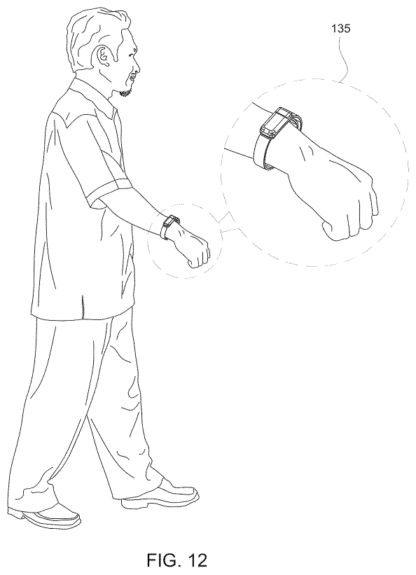

The wearable inhalation device may be designed to be worn on a user's wrist, providing convenient access for substance administration. Referring to FIG. 12, the figure illustrates an orthogonal side view showing an exemplary wrist position 135 of the wearable device. The exemplary wrist position 135 may be indicated by a dashed circular outline on the figure, demonstrating the intended placement location of the device on a user's wrist. In some cases, the wearable band may secure the device to the wrist in a manner similar to a conventional wristwatch, allowing for comfortable and discreet wear throughout the day.

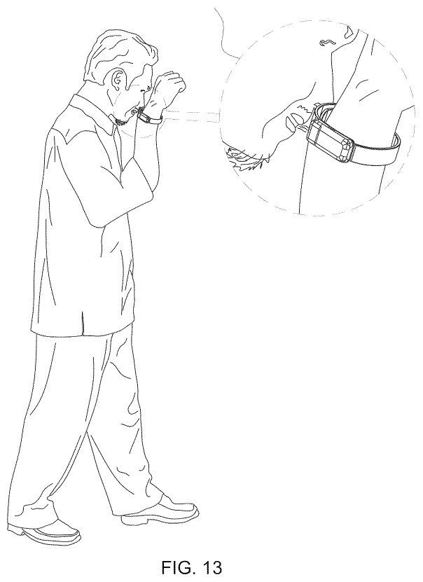

FIG. 13 illustrates a side view of a person using the wrist-mounted inhalation device. The figure shows a person in a standing position, with their arm raised toward their mouth. An inset detail circle in the upper right portion of the figure provides a magnified view of the wrist-mounted device. In some cases, the figure may demonstrate the operation of the device, showing how a user can bring their wrist to their mouth to interact with the device.

The housing cover and pod housing may be designed to resemble a typical wrist-worn accessory, allowing for discreet use in various settings. In some implementations, the airflow port may be positioned on the pod housing to facilitate easy access for inhalation when the device is worn on the wrist.

The control button may be located on the side of the housing cover, allowing users to activate or adjust the device as needed. In some cases, the indicator opening may provide visual feedback about the device's status, such as power levels or operational modes, enhancing user interaction with the device.

In some implementations, the wearable inhalation device may also function as a diffuser. The device may include an additional aperture, separate from the airflow port, through which a user can blow. This feature may allow the device to release the vaporized substance into the surrounding environment, creating an ambient effect. The diffuser function may provide an alternative method of substance administration, potentially offering calming, energizing, or therapeutic benefits to the user's immediate surroundings.

The placement and design of the wearable inhalation device may allow for seamless integration into the user's daily routine. The device's resemblance to a common wrist-worn accessory may provide discretion, while its functionality may offer convenient and controlled substance administration.

### External Design Features

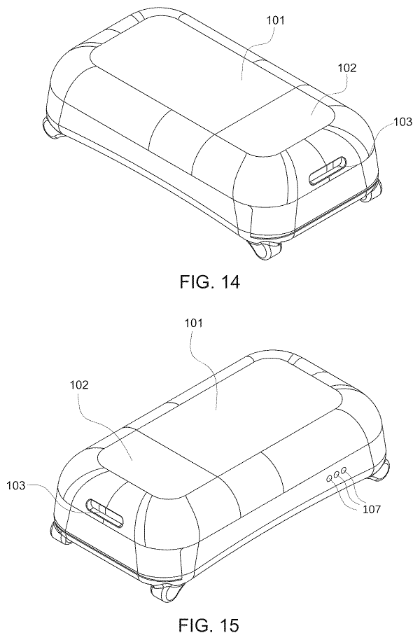

The wearable inhalation device may incorporate various external design features that contribute to both its aesthetic appeal and functional capabilities. Referring to FIGS. 14-15, the device may include the housing cover 101 that forms the outer shell of the device. The housing cover 101 may be designed with curved surfaces and smooth transitions, creating a streamlined appearance suitable for wearable applications.

In some cases, the pod housing 102 may be integrated into the housing cover 101, containing components for substance vaporization. The pod housing 102 may be positioned at one end of the device, maintaining a cohesive overall design while providing access to the substance delivery system.

The airflow port 103 may be positioned on one side of the device, as shown in FIGS. 14-15. In some implementations, the airflow port 103 may be integrated into the side profile of the pod housing 102 in a manner that maintains the device's overall aesthetic while enabling air intake or exhaust during operation.

FIGS. 14-15 may also reveal an indicator opening 107 positioned on the surface of the housing cover 101. The indicator opening 107 may allow visual feedback to be displayed to the user, potentially providing information about device status, battery life, or operational modes.

In some cases, the housing cover 101 and pod housing 102 may be designed with curved surfaces and transitions that create a streamlined appearance. This design approach may contribute to the device's discreet nature, allowing it to resemble a typical wrist-worn accessory.

The external design of the wearable inhalation device may prioritize a compact form factor suitable for wearable use. The housing cover 101 may enclose and protect the internal components while the pod housing 102 may provide access to the substance delivery system through the airflow port 103.

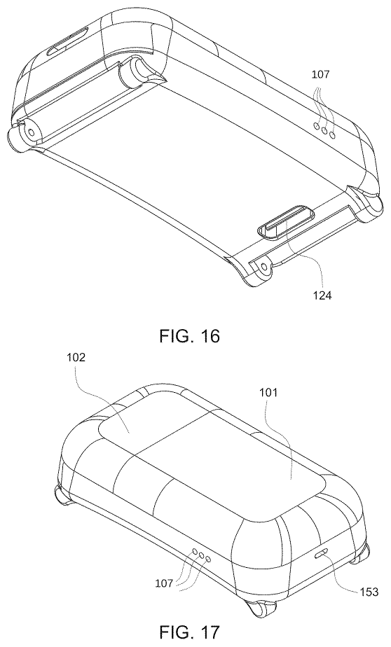

FIGS. 16 and 17 present alternative views of the wearable inhalation device, showcasing additional features and design variations. The device may comprise a body cover 101 that forms the main exterior housing, and a pod cover 102 that may be integrated into the body cover 101. An indicator opening 107 may be positioned on the surface of the device, which may accommodate visual indicators. The tank housing 124 may be visible in FIG. 16, showing how it may be incorporated into the overall structure of the device. FIG. 17 provides an alternative view that reveals a charging port 153 positioned on the device's exterior. The device may exhibit a compact form factor with rounded edges and contoured surfaces. The components may be arranged to create an integrated design where the body cover 101 and pod cover 102 form a seamless exterior, while accommodating functional elements such as the indicator opening 107, tank housing 124, and charging port 153.

In some implementations, support feet may be incorporated into the design of the device. The support feet may be positioned at strategic points on the device's base, potentially providing stability when the device is placed on a flat surface.

It may be noted that in some configurations, the control button may be optional. The device may be designed to function without a physical control button, potentially relying on other input methods such as touch sensors or motion detection for user interaction.

The external design features of the wearable inhalation device may be carefully considered to balance aesthetics, functionality, and user comfort. The streamlined form factor and integrated components may allow the device to be worn discreetly while maintaining easy access to its features and functions.

### Detailed Internal Configuration

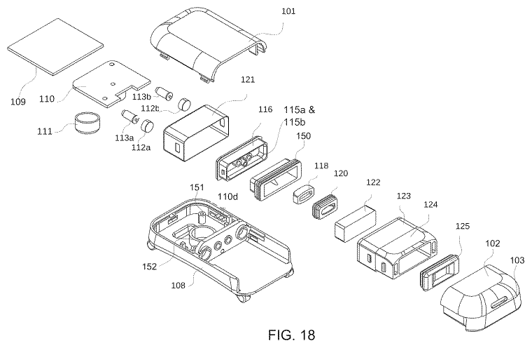

The wearable inhalation device may include a complex internal configuration designed to facilitate efficient substance vaporization and delivery. FIGS. 18-20 illustrate exploded views of the device, revealing the arrangement and interaction of various internal components.

Referring to FIG. 18, the housing cover 101 may form the outer shell of the device, connecting to the pod housing 102 which may feature the airflow port 103. Within this structure, the main enclosure 108 may serve as the primary framework for housing internal components.

The power cell 109 and circuit board 110 may be positioned near the top of the assembly. In some cases, the circuit board 110 may incorporate an indicator light 110d, which may provide visual feedback about the device's status through the indicator opening 107 in the housing cover 101.

An airflow seal 111 may be included to ensure proper air channeling within the device. The device may incorporate a magnetic connection system comprising the first magnet 112a, second magnet 112b, first male connector pin 113a, and second male connector pin 113b. These components may interface with the first female connector pin 115a and second female connector pin 115b to establish electrical connections.

The heating assembly may include the pod base 116, heating coil 118, and coil cover 120, which may be contained within the heating chamber 121. The substance reservoir 122 may connect to the reservoir base 123 and reservoir housing 124, with the pod seal 125 ensuring proper containment of the substance.

FIG. 18 also reveals a contact assembly 150, which may be integrated into a circuit housing 151. The circuit housing 151 may be mounted on a base plate 152, providing structural support for the electronic components.

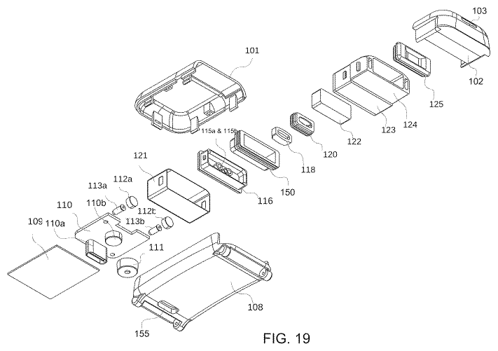

Turning to FIG. 19, additional details of the internal configuration are visible. The main enclosure 108 may be attached to a mounting bracket 155 at the bottom of the assembly. This configuration may provide stability and allow for secure attachment to the wearable band.

The circuit board 110 may include the charging port 110a and sensor module 110b. In some cases, the charging port 110a may allow for power replenishment, while the sensor module 110b may detect user interactions or environmental conditions relevant to device operation.

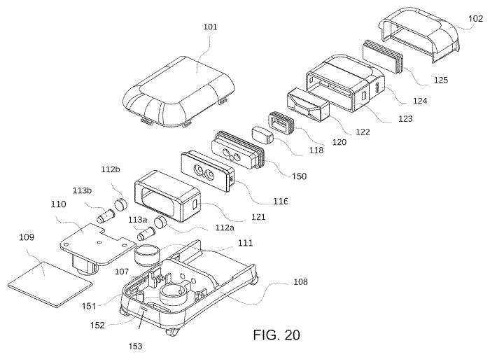

FIG. 20 provides another perspective on the internal arrangement, highlighting the relationship between components. The support feet 153 may be attached to the base plate 152, potentially providing stability when the device is not worn.

In some implementations, the arrangement of these components may allow for efficient heat transfer from the heating coil 118 to the substance in the substance reservoir 122. The compact configuration may enable the device to maintain a form factor suitable for wrist-worn use while incorporating all necessary components for substance vaporization and delivery.

The modular nature of the internal configuration, as illustrated in FIGS. 18-20, may facilitate assembly, maintenance, and potential customization of the wearable inhalation device. This design may allow for efficient operation while maintaining a compact and wearable form factor.

### Device Profile and Cross-Section

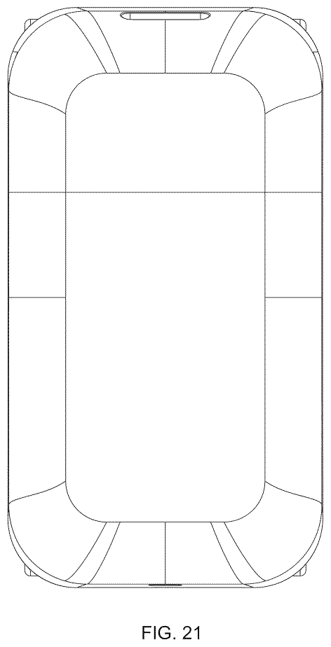

The wearable inhalation device may have a compact and streamlined profile designed for comfortable wear on a user's wrist. FIG. 21 illustrates a top orthogonal view of the device, showing its overall shape and external features. The device may have a rectangular housing with rounded corners and curved edges, creating a smooth and ergonomic form factor. The top surface of the device may include a central rectangular area that may serve as a display or interface panel. This central area may occupy a substantial portion of the top surface, potentially allowing for user interaction or information display.

In some cases, the corners of the device may incorporate curved segments that transition smoothly into the sides, enhancing the overall aesthetic and potentially improving comfort during wear. A small elongated opening or port may be visible at the top edge of the device, which may serve as an airflow port or interface for substance administration.

The housing of the device may incorporate several geometric segments and panels that create a layered appearance. These distinct sections, delineated by lines and curves that follow the device's contours, may contribute to both the aesthetic design and functional aspects of the device.

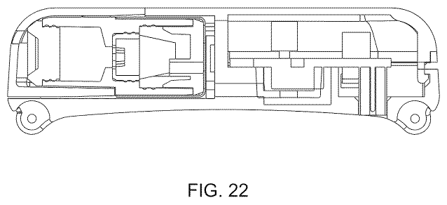

FIG. 22 provides a section view of the wearable inhalation device, revealing its internal configuration and components. The section view may show an elongated housing with rounded ends, consistent with the external profile observed in FIG. 21. The internal structure of the device may be divided into multiple compartments and chambers arranged in a linear configuration.

On one end of the sectional view, there may be a substance storage area with internal structural supports. This area may be designed to securely contain the substance intended for vaporization and administration. The middle section of the device may reveal various internal components and pathways that may facilitate substance flow and overall device operation.

The opposite end of the device may house electronic or mechanical components arranged in a compact configuration. These components may be responsible for controlling device functions, managing power, or facilitating user interaction. Two circular mounting points or supports may be visible at the bottom of the housing, potentially providing stability when the device is placed on a flat surface or interfacing with a charging mechanism.

The internal layout observed in FIG. 22 may suggest a carefully designed arrangement of components within the streamlined form factor seen in FIG. 21. This configuration may allow for efficient use of space while accommodating all necessary elements for the device's operation. The compact and integrated arrangement of components may contribute to the device's suitability for wearable applications, potentially balancing functionality with user comfort and discretion.

### Alternative Wearable Design

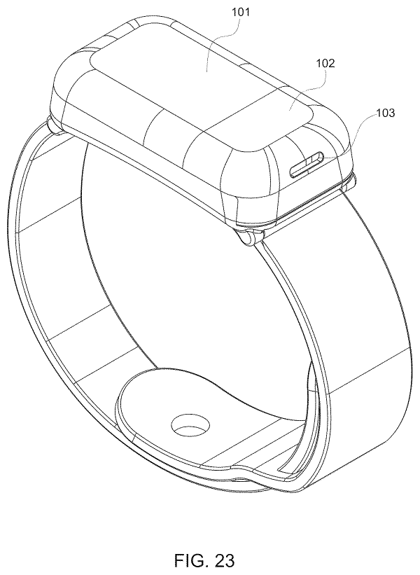

The wearable inhalation device may incorporate an alternative design that integrates the housing and band into a single, curved structure for wrist wear. Referring to FIG. 23, the device may include a housing cover 101 that forms the main body structure. In some cases, the housing cover 101 may extend to form a complete circle, creating a seamless band structure for securing the device to a user's wrist.

The pod housing 102 may be integrated into the upper portion of the housing cover 101. In some implementations, the pod housing 102 may extend above the main profile of the housing cover 101, potentially allowing for easier access to the substance delivery components.

An airflow port 103 may be positioned on the side of the pod housing 102. The placement of the airflow port 103 in this configuration may provide convenient access for inhalation when the device is worn on the wrist.

The curved design of the housing cover 101 may follow the contours of a wrist-worn accessory, potentially enhancing comfort during extended wear. In some cases, the seamless integration of the housing and band structure may eliminate the need for separate attachment mechanisms, potentially simplifying the overall design and improving durability.

The wearable inhalation device may incorporate a smart feature for tracking health data and substance usage. In some implementations, this smart feature may be integrated into the circuit board within the housing cover 101. The smart feature may include sensors for monitoring various health metrics such as heart rate, body temperature, or blood oxygen levels.

In some cases, the smart feature may also track and record substance usage data, including the type and quantity of substance consumed, as well as the frequency of use. This data may be stored within the device's memory and potentially accessed through a companion mobile application or other interface.

The integration of health tracking and substance usage monitoring may provide users with comprehensive insights into their wellness and consumption patterns. In some implementations, the device may use this data to provide personalized recommendations or alerts, potentially enhancing the user's overall health management.

The alternative wearable design, as illustrated in FIG. 23, may offer a streamlined and integrated approach to substance administration and health tracking. The combination of the curved, wrist-conforming structure with smart features may provide users with a discreet, multifunctional device that seamlessly integrates into their daily routines.

### Use as a Diffuser

In one embodiment, the method of using the WRIP device as a diffuser comprises the following steps:
1. Attaching a pod to the WRIP device: The user selects a pod containing the desired substance. The substance may be, but is not limited to, essential oils, CBD, or aromatherapy compounds. The selected pod is then securely attached to the WRIP device.
2. Exhaling or blowing into the device: The user directs their breath into the exhalation inlet of the WRIP device. The device is equipped with a mechanism to detect airflow at the exhalation inlet.
3. Substance dispersion: Upon detecting airflow, the WRIP device automatically begins dispersing the substance from the attached pod into the surrounding air. The substance is released as a fine mist or vapor, creating an ambient effect in the environment.

In some embodiments, the method may further include maintenance steps:
1. Replacing pods: When the substance in the current pod is depleted, the user removes the used pod from the WRIP device and attaches a new pod containing the desired substance.
2. Cleaning: To maintain optimal performance, the user periodically cleans the exhalation inlet of the WRIP device. This cleaning can be performed using a soft cloth or an alcohol wipe.

The method of using the WRIP device as a diffuser offers several advantages:
1. Hands-free activation: The device is triggered by the user's breath, simplifying the operation and eliminating the need for manual controls.
2. Ambient benefits: The method allows for enhancement of the surrounding environment with minimal effort, providing calming, energizing, or therapeutic effects as desired.
3. Discreet and versatile application: The WRIP device and method are suitable for use with various substances and in different settings, offering flexibility and discretion to the user.

In some embodiments, the WRIP device may include additional features such as adjustable dispersion settings, multiple pod attachments for mixing substances, or a timer function for controlled release durations.

The method described herein provides a novel and efficient way to disperse substances into the air, offering a unique combination of user-controlled activation and ambient environmental enhancement.

A number of implementations have been described. Nevertheless, it will be understood that various modifications may be made without departing from the spirit and scope of the disclosure. Accordingly, other implementations are within the scope of the following claims.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

What is claimed is:

1. A wearable inhalation device, comprising:
a housing configured to be worn on a user's wrist, the housing including a chamber for receiving a substance pod;
a modular pod system within the housing, the modular pod system configured to interchangeably receive substance pods containing various substances;
a sensor configured to detect the suction of the user's breath and activate the device;
a heating element configured to vaporize the substance within the substance pod upon activation; and
a mouthpiece in fluid communication with the chamber, the mouthpiece configured to deliver the vaporized substance to the user upon inhalation.

2. The wearable inhalation device of claim 1, wherein the housing is integrated into a wristband configured to secure the device to the user's wrist.

3. The wearable inhalation device of claim 1, wherein the sensor is an airflow sensor configured to detect the suction of the user's breath by sensing a change in air pressure within the device.

4. The wearable inhalation device of claim 1, wherein the heating element is a coil configured to heat the substance within the substance pod to a predetermined temperature to vaporize the substance.

5. The wearable inhalation device of claim 1, further comprising a power source within the housing, the power source configured to supply electrical power to the heating element.

6. The wearable inhalation device of claim 5, wherein the power source is a rechargeable battery.

7. The wearable inhalation device of claim 1, further comprising a control mechanism configured to allow the user to adjust the intensity of the vapor produced by the device.

8. A method for administering substances using a wearable inhalation device, comprising:
providing a wearable inhalation device having a housing configured to be worn on a user's wrist, the housing including a chamber for receiving a substance pod;
inserting a selected substance pod into a modular pod system of the device, the substance pod containing a desired substance;
activating the device in response to detecting the suction of the user's breath via a sensor;
vaporizing the substance within the substance pod using a heating element; and
delivering the vaporized substance to the user through a mouthpiece in fluid communication with the chamber upon inhalation.

9. The method of claim 8, wherein the substance pod contains a substance selected from the group consisting of CBD, THC, melatonin, caffeine, and combinations thereof.

10. The method of claim 8, further comprising a step of recharging a power source within the housing, the power source configured to supply electrical power to the heating element.

11. The method of claim 8, wherein the sensor is an airflow sensor configured to detect the suction of the user's breath by sensing a change in air pressure within the device.

12. The method of claim 8, further comprising a step of adjusting the intensity of the vapor produced by the device using a control mechanism.

13. The method of claim 12, wherein adjusting the intensity of the vapor includes modifying at least one of: temperature of the heating element, duration of heating, or airflow rate through the device.

14. The method of claim 13, wherein the control mechanism includes a touch-sensitive surface integrated into the housing of the device.

15. A wearable inhalation device, comprising:
a housing configured to be worn on a user's wrist, the housing including a chamber for receiving a substance pod;
a modular pod system within the housing, the modular pod system configured to interchangeably receive substance pods containing various substances;
a sensor configured to detect the suction of the user's breath and activate the device;
a heating element configured to vaporize the substance within the substance pod upon activation;
a mouthpiece in fluid communication with the chamber, the mouthpiece configured to deliver the vaporized substance to the user upon inhalation; and
a smart feature configured to provide health tracking functionalities and substance usage data to the user.

16. The wearable inhalation device of claim 15, wherein the smart feature is configured to track and display the user's substance usage data, including the type of substance used, the quantity of substance used, and the frequency of use.

17. The wearable inhalation device of claim 15, wherein the smart feature is further configured to provide health tracking functionalities, including tracking the user's heart rate, oxygen level, and body temperature.

18. The wearable inhalation device of claim 17, wherein the smart feature is further configured to provide alerts to the user based on the health tracking data, the alerts including reminders to administer the substance at predetermined intervals or warnings when abnormal health data is detected.

19. The wearable inhalation device of claim 15, further comprising a wireless communication module configured to transmit the health tracking functionalities and substance usage data to a mobile device or cloud-based platform.

20. The wearable inhalation device of claim 19, wherein the wireless communication module is further configured to receive updates and customized settings for the device from the mobile device or cloud-based platform.
